(19) United States
(12) Patent Application Publication
(10) Pub. No.: US 2005/0118115 A1
(43) Pub. Date: Jun. 2, 2005

(54) Accelerated tooth whitening method, composition and kit

(76) Inventor: Mark G. Fontenot, Palo Alto, CA (US)

Correspondence Address: Howard M. Peters;PETERS, VERNY, JONES & SCHMITT, LLP, Suite 6, 385 Sherman Avenue, Palo Alto, CA, 94306, US

(21) Appl. No.: 10/693,088

(22) Filed: Oct. 24, 2003

(51) Int. Cl. A61K 7/20

(52) U.S. Cl. 424/53

(57) ABSTRACT

The present invention concerns an improved method and different compositions to whiten and to accelerate the whitening of a tooth. The invention utilizes a primer of a hypochlorite and tooth whitening agent of a peroxide in combination which react on the surface of the tooth being whitened. Optionally the primer includes a flavoring agent. Various kits of the components of the invention are disclosed.

### BACKGROUND OF THE INVENTION

1. Field of Invention

The present invention relates to an improved method and composition to accelerate tooth whitening. It also includes the method's use in diagnosis for continued tooth whitening treatment, response to tooth whitening treatment, and a kit of components for in-office or at-home tooth whitening.

2. Description of the Background and Related Art

Smiling is the end result of a complex social, neurologic, psychological process. Because a smile is universally understood, an unattractive smile, due in part to tooth staining, may have negative psychological and social implications. If tooth staining is left untreated, it may affect the appearance of a person's smile and self-perception, causing temporary, as well as permanent, social and psychological sequelae.

A tooth is composed of a crown and one or more roots. The crown is the portion of the tooth exposed within the oral cavity and the root anchors the tooth in the bone of the maxilla or mandible. The transverse section of the crown has a white enamel outer layer which is supported by the underlying dentin. The transverse section of the root has a cementum outer layer which is supported by dentin underneath. The pulp chamber forms the core of the root and crown and contains nerves, arteries, and veins which supply sensory and metabolic nutrition to the tooth.

Enamel is white to off-white in color and covers the surface of the crown. Enamel is a hard, biologic composite material consisting predominantly of an inorganic hydroxylapatite crystalline matrix and approximately 5% organic material in the form of collagen. Dentin within the tooth structure is yellowish in color and softer than enamel. Dentin is also a composite of biologic materials consisting of about 20% protein including collagen, and the balance consists of the inorganic material in the form of hydroxyapatite. In the oral cavity, the clinical crown is covered with proteinaceous layer called the acquired pellicle, which is a viscous composite biological structure consisting of saliva, glycoproteins, bacteria, minerals, and other compounds and components.

Tooth staining is caused by multiple local and systemic influences that can alter the color of the acquired pellicle, enamel, and dentin. Stain distribution patterns vary from localized (1 or 2 teeth) to regional or generalized involvement of teeth. No sex or racial predilection exists for tooth staining. Teeth generally become more yellow and gray with increasing age. Depending on the etiology, persons of different ages are susceptible to various types of internally induced and externally induced tooth discoloration. Staining of teeth can be generally categorized as staining that occurs from extrinsic and/or intrinsic factors.

Extrinsic dental stains are caused factors such as dental plaque and calculus that accumulates on the teeth, diet and the intake of foods and beverages, social habits including the use of tobacco and alcohol, chromogenic bacteria, metallic compounds, and topical oral rinses and medications. Extrinsic staining results in tooth colors that have a brown, black, gray, green, orange, and/or yellow characteristic. In some cases, the scratch test is usually used to diagnose extrinsic tooth staining. Extrinsic staining of one tooth is unusual; however, it is generally found on tooth surfaces with poor toothbrush accessibility. For example, extrinsic tooth staining occurs frequently at the tooth-gingival interface (cervical regions) and between the teeth (interproximal regions). Generally, extrinsic tooth staining begins in the acquired pellicle from tannins, polyphenolic, and other staining compounds resulting from diet, social habits, and topical medications that become trapped in and tightly bound to the organic components of the pellicle.

Intrinsic dental stains are caused by a number of different factors including but not limited to dental materials used in the restoration of teeth, dental conditions such as periodontal disease and caries, trauma to the teeth and dental arches, infections of the periodontium, mandible, and maxilla, systemic medications such as tetracylines, nutritional deficiencies, disorders such as complications of pregnancy, anemia and bleeding disorders, bile duct problems, and genetic defects and hereditary diseases, for example, those affecting enamel and dentin development or maturation. Tooth staining as a result of intrinsic factors contributes to tooth discoloration having a brown, black, gray, green, orange, and/or yellow appearance. Unlike extrinsic staining, teeth with intrinsic discoloration may be red or pink. A diagnostic test used to distinguish intrinsic from extrinsic stains is illumination of teeth using ultraviolet light causing tetracycline staining or congenital porphyria to fluoresce yellow or red, respectively. Intrinsic discoloration cannot be removed by using the scratch test. The tooth distribution of intrinsic staining is either generalized to all teeth or localized to certain teeth or tooth surfaces. An intrinsic etiology usually exists when a single tooth is discolored. When multiple teeth are involved, patterns of banding are indicative of intrinsic staining.

Dental treatment of tooth stains involves identifying the etiology and implementing therapy. In some cases, medical treatment also may be warranted, depending on the etiology of the tooth discoloration. The topical application of peroxide gels and solutions to teeth is commonly used to whiten teeth. Peroxides are oxidizing agents have been found conventionally to be safe and effective. However, approximately 50% of patients using peroxides to whiten teeth may have short term side effects that are completely reversible in 1 to 4 days.

Commonly used peroxides to whiten teeth include hydrogen peroxide H₂O₂ (HP), carbamide peroxide (CP), sodium perborate (NaBO₃), and sodium percarbonate (NAHCO₄). HP and CP are two of the most widely used peroxides. Typically, HP tooth whitening gels are available in concentrations ranging approximately from 3% to 35%, with the latter limited to professional application for in-office power tooth whitening or power bleaching. CP is commercially available in concentrations ranging approximately from 10% to 30%. When in contact with teeth, peroxides oxidize organic stains in the acquired pellicle, enamel, and dentin which results in whiter looking teeth. However, teeth stained as result of inorganic compounds tend not to respond to conventional peroxide tooth whitening gels. The tooth whitening dose response to peroxides is wide and variable. Generally, a great majority of patients will experience some level of tooth whitening when peroxides are applied to the teeth for adequate periods of time and within clinically acceptable peroxide concentrations.

There are three general methods to deliver peroxide gels to the teeth, namely 1) paint-on, 2) tray-and-gel, and 3) strips. Professional paint-on methods include in-office chair side power bleaching wherein 25% or greater hydrogen peroxide gels are professionally applied to the facial surfaces of teeth using a brush. Some of these professional paint-on systems work in conjunction with light or heat energy. Over the counter (OTC) paint-on systems such as Colgate Simply White™ (a trademark of the Colgate Palmolive Corp., New York, N.Y.) consist of 18% carbamide peroxide solutions and are applied to the facial surfaces of the teeth by the consumer.

Professional custom tray-and-gel methods consist of custom fabricated dental trays wherein a reusable dental tray is fabricated specifically to the dental arch of the patient. The dental professional dispenses several 3-ml syringes of approximately 6% to 9% hydrogen peroxide gel or approximately 10% to 30% carbamide peroxide gel. On a daily basis, the patient dispenses tooth whitening gel from the syringe into the custom dental tray, then places the dental tray over the dental arch for one to two hours or longer. After each daily treatment, the tray is removed, cleaned, and stored for future use (See for example U.S. Pat. Nos. 5,098,303; 5,234,342; 5,409,631; 5,725,843; and 6,183,251).

Over the counter (OTC) reusable tray-and-gel methods consist of a generic, universal reusable dental tray and 4 to 6 syringes containing approximately 3 ml of approximately 5% hydrogen peroxide or approximately 10% to 15% carbamide peroxide. The consumer dispenses gel from a syringe into the reusable dental tray and places it over the arch for 30 minutes to one hour daily. After each daily treatment, the reusable dental tray is removed from the dental arch, cleaned, and stored for future use (See U.S. Pat. No. 5,165,424). Professional and OTC disposable tray-and-gel methods consist of disposable dental trays pre-filled with peroxide gel. The patient or consumer places the disposable tray with the tooth whitening gel pre-dispensed into the tray over the dental arch daily for 30 minutes or longer. After treatment, the tray is removed and discarded (See for example U.S. Pat. Nos. 5,575,654; 5,863,202; and 5,980,249 and assigned to OraPro, Inc., Palo Alto, Calif.).

The strip method used a thin polymer strip with a thin layer of peroxide tooth whitening gel laminated to one side. The strip method is offered by Crest Whitestrips™ (a trademark of the Proctor & Gamble Corp., Cincinnati, Ohio) and is available as an OTC or professional system. Whitestrips are thin, flexible polyethylene strips that have a coating of tooth whitening gel on one side. The peroxide gel side of the strip is placed against the facial surfaces of the anterior teeth (See for example U.S. Pat. Nos. 5,891,453; 5,879,691; and 6,277,458).

The general efficacy of over-the-counter (OTC) peroxide tooth whitening is found in Table 1. Colgate Simply White™ is an example of a peroxide tooth whitening product that uses the paint-on method and containing a solution of 18% carbamide peroxide (CP). The average shade improvement using Colgate Simply White™ is on the order of 2 shades after 7 hours of treatment time in a two-week period.

Crest White Strips™ are peroxide tooth whitening products that use the strip method to deliver peroxide tooth whitening gels to the facial surfaces of teeth. For the OTC markets, Crest WhiteStrips™ is offered in 5.5% HP. For the professional dental markets, Crest WhiteStrips™ are offered in percent concentrations up to 14% HP. It has been reported that Crest WhiteStrips™ for the OTC market (5.5% HP) can whiten teeth on order of 4 shades after 14 hours of treatment time in a two-week period.

TABLE 1
Summary of over-the-counter peroxide tooth whitening methods for
paint-on, strips, and tray-and-gel method of delivering peroxide
tooth whitening gelsand solutions

| | Products | | | |
|---|---|---|---|---|
| | Colgate | | | |
| | Simply | Crest | Rembrandt | |
| Parameter | White ®^{a} | WhiteStrips ®^{b} | Denmat ®^{c} | OraPro^{d} |
| Delivery | Paint-on | Strip | Reuseable | Disposable |
| method | | | tray-and-gel | tray-and-gel |
| Whitening gel | CP (18%) | HP (5.5%) | CP (10%) | HP (6%) |
| (CP or HP) | | | | |
| Estimated tooth | 2 | 4 | 6 | 6 |
| shade | | | | |
| improvement | | | | |
| Average | 14 | 14 | 14 | 14 |
| number of | | | | |
| treatment days | | | | |
| Number of | 2 | 2 | 2 | 2 |
| daily | | | | |
| treatments | | | | |
| Ave. daily | 30 | 30 | 30 | 30 |
| treatment | | | | |
| duration (min) | | | | |
| Total treatment | 14 | 14 | 14 | 14 |
| time (hours) | | | | |
| Treatment time | 7.0 | 3.5 | 2.3 | 2.3 |
| (hour) per | | | | |
| shade | | | | |

^{a}Trademark of Colgate Palmolive Corp., New York, New York
^{b}Trademark of Proctor & Gamble Corp., Cincinnati, Ohio
^{c}Trademark of Denmat Corp., Santa Maria, California
^{d}OraPro, Inc., Palo Alto, California

OTC tray-and-gel methods are delivered using a reusable dental and peroxide tooth whitening gel supplied in syringes or disposable dental trays pre-filled with peroxide tooth whitening gels. In the former, OTC reusable tray-and-gel methods are offered, for example, by DenMat Corporation (Santa Maria, Calif.) under the Rembrandt™ brand name. Peroxide tooth whitening gel concentrations are about 10% CP. The reported use of OTC reusable tray-and-gel methods can whiten teeth on the order of 6 shades after 14 hours of treatment time in a two week period.

OTC disposable tray-and-gel methods invoke a single use, disposable dental tray pre-filled with 6% HP. The reported use of OTC disposable, single-use tray-and-gel methods can whiten teeth on the order of 6 shades after 14 hours of treatment time in a two week period.

The difference in the rate of tooth whitening is characteristic of a dose response which is related to the concentration and quantity of peroxide tooth whitening gel delivered to the teeth, frequency and duration of treatment, susceptibility of tooth staining to peroxide oxidation, and chemical dynamics of oxidizing tooth stains. Generally, paint-on methods provides the least tooth whitening efficacy, strip methods are moderately effective, and tray-and-gels methods are most effective. Efficacy for these methods ranges from 2 shades to 6 shades with 14 hours of treatment time in a two week period.

The most effective tooth whitening methods are those delivered by dental professionals to their patients. Custom fabricated tray-and-gel methods deliver about 6 shades or greater of improvement with about 14 hours of treatment time in a two-week period. Dentists can dispense a variety of concentrations and types of peroxide tooth whitening gels to their patient. The most effective method to whiten teeth is professionally applied paint-on peroxide tooth whitening gel which is commonly referred to as power bleaching. These professional paint-on methods used HP gels with concentrations up to 35%. Generally, the shade improvement after a one-hour session of in-office power bleaching using the paint-on method with 35% HP is on the order of 6 to 8 shades or greater in some cases. However, a significant percentage of the patients receiving in-office power bleaching treatment can have tooth sensitivity to cold and soft tissue irritation resulting from the high concentration of HP tooth whitening gel.

Historically hypochlorite solutions are used in some limited dental procedures. For example, hypochlorite is used to remove organic material in a area abraded from a tooth structure prior to a filling or crown replacement (U.S. Pat. Nos. 4,850,872; 6,582,681), and for sterilizing dental instruments (U.S. Pat. No. 6,207,201). A recent publication generally describes a chemically curing solid dental bleaching material. (U.S.P.T.O. Patent Publication 20020197214.) Hypochlorite solutions for dental applications are sometimes difficult to use and to quantify. Hypochlorite solutions normally have a taste and an odor that are disagreeable and irritating to most dental patients.

Additional references of interest in this include, but are not limited to, the following:
U.S. Pat. Nos. 3,339,547; 3,527,219; 4,060,600; 4,138,814; 4,226,851; 4,661,070; 4,687,663; 4,788,052; 4,952,143; 4,968,251; 4,983,380; 4,983,831; 4,990,089; 5,009,885; 5,032,178; 5,199,567; 5,240,415; 5,326,685; 5,346,061; 5,356,291; 5,376,006; 5,339,547; 5,571,519; 6,077,502; 6,350,438; and 6,610,276.

All patents, patent applications, articles, references, standards, commercially available materials and the like which are cited in this application are incorporated by reference in their entirety. The present invention is not taught or suggested by the art cited herein.

From the above discussion it is apparent that a need still exists for improved methods, compositions and kits of various components for the accelerated whitening of a tooth. The present invention describes a number of advances.

### SUMMARY OF THE INVENTION

The present invention is directed toward an accelerated tooth whitening composition and method wherein the tooth surface primer of the present invention is applied to the surface of the tooth, after which a peroxide tooth whitening composition is delivered and applied to the teeth. The primer is a sodium hypochlorite composition (e.g. sodium or calcium) with a high pH that penetrates and disrupts the acquired pellicle, and penetrates the surface of the enamel and into the sub-surface of the enamel of the tooth and begins to oxidize organic stains through oxidation resulting in whiter teeth. When peroxide tooth whitening gel is delivered to the tooth, a pH gradient is immediately established between the primer of the present invention and the peroxide tooth whitening gel. Mixing of the peroxide tooth whitening gel and the primer of the present invention occurs at the surfaces of teeth as a result of physical, mechanical and chemical factors including, but not limited to, delivery of the peroxide tooth whitening gel to the dental arch, gross and micro-movement of the peroxide tooth whitening gel relative to the primer of the invention, and mixing that occurs as a resulting of the pH gradient and swelling pressures between to primer and peroxide tooth whitening gel and combination thereof (includes reactivity). The pH differential causes intense coupled oxidation processes of the sodium hypochlorite primer of the present invention and the peroxide tooth whitening gel. The oxidation processes occur at the surface and sub-surface of the enamel of the tooth as well as the dentin, which collectively is the primary staining zone of teeth. Immediate oxidation process occurs with the application of the hypochlorite primer alone to the surface of the tooth and enhanced oxidation occurs between the primer of the present and peroxide tooth whitening gels in the tooth staining zone of tooth which is the acquired pellicle, surface of the enamel, and sub-surface of the tooth structure. Rapid and enhanced oxidation following the administration of the primer of the present invention and peroxide tooth whitening gels to the surface of teeth results in accelerated tooth whitening.

The present invention relates to a primer having an alkaline based composition which is formulated from sodium hypochlorite, sodium hydroxide, potassium hydroxide, calcium hydroxide and combinations thereof. The primer is applied to the surface of the teeth before the application of peroxide tooth whitening gels using a variety of delivery methods. These include spray, brushing, strips, trap and the like, and include combinations thereof. The primer can be an aqueous solution or a gel.

The present invention further relates to a method for applying the primer to the surfaces of the teeth to disrupt the pellicle, increase the pH of the surface and sub-surface of the teeth, and in one embodiment initiate tooth whitening via chlorine oxidation of tooth stains, after which any of a number of methods to delivering peroxide tooth whitening compositions are invoked for the treatment of stains comprising the steps of:
(a) obtaining a stable high pH primer solution from sodium hypochlorite having a pH greater than about 8.5 up to about 13;
(b) applying the primer solution to the teeth;
(c) delivering tooth whitening compositions to the tooth comprising peroxide. This application to the dental arch may occur using paint-on, strips, and tray-and-gel delivery methods, and combinations of these methods, followed by
(d) removing the components.

In another embodiment of the present invention, the primer is selected from the group consisting of sodium hypochlorite, potassium hypochlorite, calcium hypochlorite, barium hypochlorite and combinations thereof.

In another embodiment of the present invention, the hydroperoxide is selected from the group consisting of hydrogen peroxide, sodium peroxide, potassium peroxide, carbamide peroxide, sodium perborate, sodium percarbonate, and combinations thereof.

In another embodiment, the present invention comprises a kit of components and applications means including instructions for application for the care giver or patient.

In another embodiment, the present invention comprises a method useful in observing the tooth whitening treatment response and in diagnosing tooth staining, which assists in the determination of subsequent tooth whitening therapy.

In another embodiment, the present invention concerns a method which include a primer comprising sodium hypochlorite and a peroxide comprising hydrogen peroxide.

In another embodiment, the present invention concerns a composition which includes a primer comprising sodium hypochlorite, a peroxide comprising hydrogen peroxide and optionally an aqueous, gel or foam fluoride treatment.

In another embodiment the present invention concerns a kit which comprises a primer comprising hypochlorite, a primer applicator, and instructions on which kit is useful as a pre-treatment prior to using a subsequent tooth whitening composition comprising peroxide. In these embodiments, the following articles are optionally present individually or in any combination with each other: tooth whitening composition, tooth whitening applicator, desensitizing toothpaste, tooth brush and the like.

### BRIEF DESCRIPTION OF THE DRAWINGS

The advantages of the present invention will become apparent from the following detailed description of the invention, which should be read in conjunction with the following drawings.

FIG. 1 is a schematic representation of a transverse section of a dental arch showing the application of the primer of the present invention to the facial surface of a tooth.

FIG. 2 is schematic representation of a transverse section of a dental arch showing the primer on the facial surface of a tooth.

FIG. 3 is a schematic representation of a transverse section of a dental arch showing a layer of peroxide whitening solution that has been applied over a layer of the primer. The primer was applied first followed by the application of the peroxide tooth whitening solution using the paint-on method.

FIG. 4 is a schematic representation of a transverse section of a dental arch showing a strip having a layer of peroxide tooth whitening gel in contact with a layer of primer. The strip consists of a thin polymer film with peroxide tooth whitening gel laminated to the tooth-side of the strip. The primer of the invention was applied before the strip was placed on the facial surfaces of the teeth in the manner shown.

FIG. 5A is a schematic representation of a transverse section of a custom dental arch showing a tray filled with peroxide tooth whitening gel that is placed over the dental arch with the gel contacting the layer of primer on the facial surface of the tooth. The primer was applied first followed by the application of the tray and gel over the teeth of the dental arch. FIG. 5B is a schematic representation of a transverse section of a reusable or disposable tray and gel.

FIGS. 6A and 6B are photographs of teeth whitened by the present invention. FIG. 6A is before whitening and FIG. 6B is after whitening using sodium hypochlorite and hydrogen peroxide as described in Example 2.

FIG. 7A is a schematic representation of a professional in-office kit and its components of this invention and FIG. 7B is a top plan view of the kit. FIG. 7C is a cutaway schematic representation of the individual container of the primer.

FIG. 8 is a schematic representation of an over-the-counter (at-home) kit and its components of this invention.

FIG. 9 is a schematic representation of the treatment option flow chart. In the therapy, patients are triaged (provided treatment options) using the primer (SH) combined with the dispersable dental tray with 10% hydrogen peroxide.

### INVENTION AND DETAIL DESCRIPTION OF THE PREFERRED EMBODIMENTS

Definitions

As used herein and optionally described elsewhere in this application:

“Enamel” refers to the white hard biologic composite after surface of the tooth. It consists predominantly of hydroxylapatite.

“Flavoring” or “flavoring agents” refers to, but is not limited to mint, oil of wintergreen, oil of peppermint, oil of spearmint, clove bund oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof. Flavoring agents are generally used in the compositions at levels of from about 0.001% to about 5% by weight of the composition.

“Fluoride” refers to the conventional fluoride dental treatments.

“Kit” refers to the components of this invention for use in-office or at-home tooth whitening.

“OTC” refers to pharmaceuticals and devices which are sold over-the-counter under the regulations of the U.S. Food and Drug Administration if applicable.

“Pellicle” refers to a viscous composite biological structure which comprise saliva, glycoprotein, bacteria, minerals and other compounds and components known in the dental art.

“Peroxide” refers to hydrogen peroxide, sodium peroxide, potassium peroxide, carbamide peroxide, sodium perborate, sodium percarbonate and combinations thereof. The pH of the primer is between about 8.5 and 13, preferably between about 8.5 and 12, more preferably between about 8.5 and 11 and even down to about 10.

“Primer” refers to a hypochlorite solution as is described herein. Sodium hypochlorite is preferred.

“Substantially” refers to a situation wherein an exact correlation is not observed by that 80-90% similarly is present.

“Synergistic” refers to a situation wherein each component above has a specific effect and in combination that two effects are at least 10% greater than the effect of the individual component.

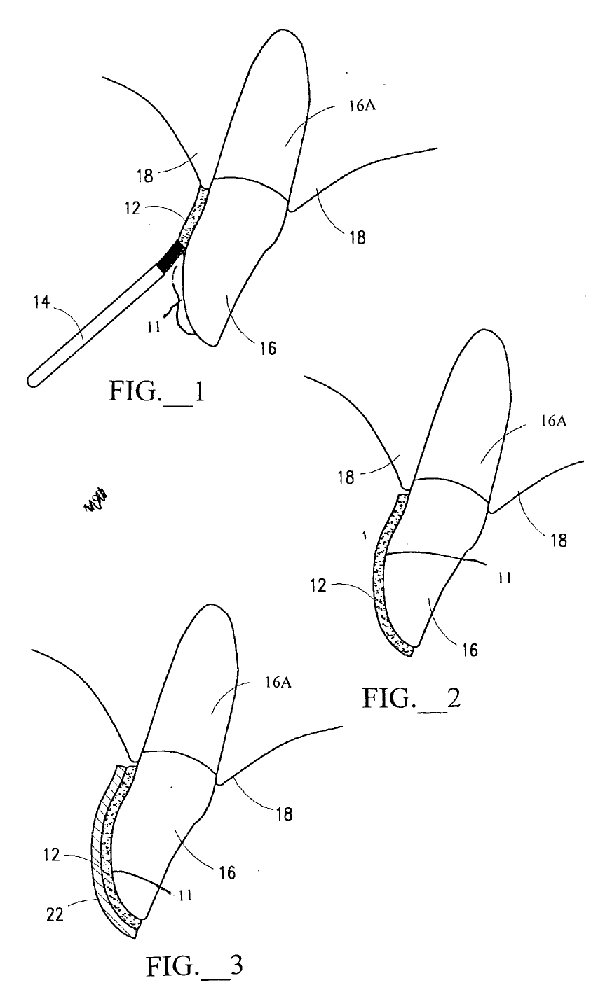

The primer solution shown in FIG. 1 is being applied to the facial surface of teeth using an applicator brush. The primer of the present invention is composed of an alkaline solution or gel of sodium hypochlorite, sodium hydroxide, potassium hydroxide, calcium hydroxide or similar pH modifier (Table 2), a viscosity thickening agent such as CARBOPOL® or METHOCEL® to form a viscous gel, water, alcohol, and glycerin. The thickening agent should be relatively inert and increase the viscosity of the primer and the shear forces required to cause flow when the primer is applied to the facial surfaces of the teeth.

TABLE 2
Compositional ranges of various constituents used in the primer.

| Ingredient | Weight Percent |
|---|---|
| Purified Water | 30% to 70% |
| Alcohol | 10% to 20% |
| Glycerine | 5% to 10% |
| pH Modifier* (e.g. SH) | 0.1% to 15% |
| Thickening Agent | 1% to 10% |

A large number of commercially available peroxide tooth whitening gels are stabilized at a pH ranging from 3 to approximately 7. When peroxide tooth whitening gels such as HP are placed in contact with high pH solutions or gels such as the primer of the present invention, destabilization of the HP gel ensues and hydroxyl and perhydroxyl free radicals are released as well as oxygen gas. The primer thus acts to drive the reaction of HP. In particular, when sodium hypochlorite primer of the present invention is used and placed in contact with peroxide tooth whitening gels, a dual tooth whitening oxidation process ensues between the hypochlorite, such as sodium hypochlorite (SH) and the peroxide tooth whitening gel.

The (SH) primer of this invention can be a flavored solution (e.g. mint, cinnamon, etc.) having the SH at pH of approximately 12. SH is conventionally used in a number of clinical dental applications including bacterial control in halitosis, root canal irrigant, and topical tooth whitener. Clinically, SH exhibits an antimicrobial effect which is related to the inherently high pH (hydroxyl ions action). As an endodontic irrigant, sodium hypochlorite acts as an organic solvent, by degrading fatty acids into fatty acid salts (soap) and glycerol (alcohol), which reduces the surface tension of the remaining solution (saponification reaction). A topical solution of 5% SH has been successfully used to whiten hypomineralized yellow-brown stains using 1 to 2 applications (10 to 15 minutes each) (Wright, American Assoc. Pediatric Dentistry, 2003).

The primer is used in combination with any method of applying peroxide-based tooth whitening gel to the surfaces of teeth. For example, Colgate Simply White™ is composed of 18% CP tooth whitening solution and is applied to the facial surfaces of the teeth using an applicator brush coupled to the cap. To accelerate tooth whitening using the Colgate Simply White™ paint-on product, the SH primer of the invention is first applied to the facial surfaces of the teeth using an applicator such as a brush (see FIGS. 1 and 2). The SH primer has a high pH. Next, the Colgate Simple White™ solution is applied over the primer layer and to the facial surfaces of the teeth as per the manufacturer's instructions (FIG. 3). Application of the primer to the facial surfaces of teeth accelerates tooth whitening by disrupting the acquired pellicle and establishing an elevating the pH of the environment at facial surface and sub-surface of the teeth. As the SH primer is initially applied to the facial surfaces of the teeth, the SH begins to oxidize tooth stains and whiten teeth immediately. The primary staining zone of teeth is located in the acquired pellicle on the facial surface of the teeth and the facial enamel surface and facial enamel sub-surfaces of the teeth and into the facial surface of the dentin. After application of the CP based Colgate Simply White™ tooth whitening solution, destabilization of the peroxide gel ensues and hydroxyl and perhydroxyl free radicals are released as well as oxygen gas. When placed on the tooth surface, the primer acts to enhance and drive the reaction of peroxide tooth whitening gels to rapidly oxidize stains and accelerate the tooth whitening process.

In the professional dental setting of the dental office, the SH primer is used in conjunction with in-office power bleaching methods that use high concentrations of peroxide gels in the range of 20% to 35% HP. The soft tissue of the oral cavity (18) is isolated using a rubber dam, for example, so that the hard tissue facial surfaces (11) of the tooth (16) having root (16A) are isolated from the adjacent soft tissue. The primer (12) of the present invention (e.g. SH) is first applied to the facial surfaces (11) of the tooth (16) using a single use, disposable applicator brush (14) shown in FIGS. 1 and 2. As per the manufacturer's instruction, the in-office power bleaching gel (22) is then applied over the primer (12) and on the facial surfaces (11) of the tooth (16) (FIG. 3). The SH primer has a high pH. The SH primer accelerates tooth whitening by disrupting the acquired pellicle and establishing a pH environment at facial surface and sub-surface of the teeth in which the primer of the invention was applied. The primary staining zone of teeth is located in the acquired pellicle on the facial surface of the teeth and the facial enamel surface and facial enamel sub-surfaces of the teeth and into the facial surface of the dentin. After application of the in-office power bleaching gel (22) over the primer (12), destabilization of the peroxide gel ensues and hydroxyl and perhydroxyl free radicals are released as well as oxygen gas. When placed on the tooth surface, the primer (12) acts to enhance and drive the reaction of peroxide tooth whitening gels (22) to rapidly oxidize stains and accelerate the tooth whitening process. Additionally, the SH primer begins to oxidize tooth stains and whiten teeth immediately after applying to the teeth.

Strips consist of a thin polymer film with a layer of peroxide tooth whitening gel laminated to one side. The use of strips has become a popular method to deliver peroxide tooth whitening gels to the facial surfaces of teeth. Crest WhiteStrips™ manufactured by Procter & Gamble uses a strip method to deliver peroxide tooth whitening gels to the facial surfaces of teeth to whiten teeth. Crest WhiteStrips™ offers several different concentrations of hydrogen peroxide gel ranging from approximately 5% for an over-the-counter product to 14% hydrogen peroxide gel for professionally dispense tooth whitening product.

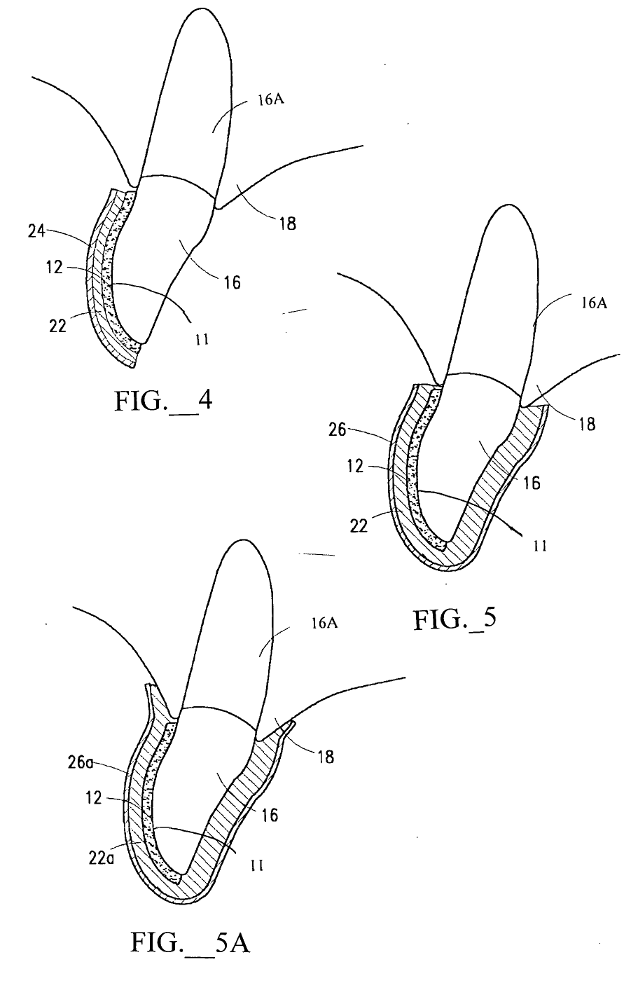

The use of the primer invention can be used with in conjunction with any of the Crest WhiteStrips™ formulations. To accelerate tooth whitening using the strip method such as that offered by Crest WhiteStrips™ in combination with the invention, the SH primer of the invention is first applied to the facial surfaces of the teeth using an applicator such as a brush (FIGS. 1 and 2). Next, Crest WhiteStrips™ is applied over the primer layer and on the facial surfaces of the teeth as per the manufacturer's instructions (FIG. 4). After application of the Crest WhiteStrips™ over the facial surfaces of the teeth, destabilization of the hydrogen peroxide gel ensues and hydroxyl and perhydroxyl free radicals are released as well as oxygen gas. When placed on the tooth surface, the primer acts to enhance and drive the reaction of peroxide tooth whitening gels to rapidly oxidize stains and accelerate the tooth whitening process. The SH primer begins to oxidize tooth stains and whiten teeth immediately after applying to the teeth.

Tooth whitening gels can also be delivered to the dental arch using dental trays. These methods are referred to tray-and-gel methods. In one approach using a tray-and-gel method, the dentist can fabricate a custom dental tray for a patient by taking an impression of the dental arch and making a reproduction of the dental arch from the impression using dental stone. Using a vacuum heat method, a thin polymer dental tray is fabricated to the stone model of the patient's dental arch. The polymer tray is trimmed and fitted to the teeth. The dentist dispenses several syringes (e.g. 3 ml) of peroxide tooth whitening gel. The patient is instructed to dispense peroxide tooth whitening gel in the trough of the tray and place the custom dental tray daily over the dental arch for 30 minutes to overnight treatment. After treatment, the custom dental tray is removed, cleaned, and stored for future use. The process is repeated daily for several weeks. The primer of the invention can be used in conjunction with a custom dental tray and gel method to accelerate the tooth whitening process. The SH primer of the invention is first applied to the facial surfaces of the teeth using an applicator such as a brush (FIGS. 1 and 2). After application of the SH primer, the patient places the custom dental tray with peroxide tooth whitening gel over the dental arch as per the dentist's instructions. The primer accelerates tooth whitening by disrupting the acquired pellicle and establishing a pH environment at facial surface and sub-surface of the teeth in which the primer of the invention was applied. After application of the custom dental tray with peroxide tooth whitening gel to the dental arch, destabilization of the hydrogen peroxide gel ensues and hydroxyl and perhydroxyl free radicals are released as well as oxygen gas. When placed on the tooth surface, the primer acts to enhance and drive the reaction of peroxide tooth whitening gels to rapidly oxidize stains and accelerate the tooth whitening process. The SH primer begins to oxidize tooth stains and whiten teeth immediately after applying to the teeth.

In another tray and gel method available in the over-the-counter market, a reusable dental tray is available with several syringes of peroxide tooth whitening gel, such as those Rembrandt® products offered by DenMat Corp, Santa Maria, Calif. The consumer may custom fit the reusable dental tray by dipping the polymer reusable dental tray into warm water the placing the dental tray over their dental arch. The tray is removed and an impression of the dental arch is made in the reusable dental tray. To begin the tooth whitening process, the consumer dispenses gel into the tray and places the tray over the dental arch. Typical recommended daily treatment times range from 30 minutes to two hours. After daily treatment, the tray is removed, cleaned, and stored for future use.

The primer of the present invention can be used in conjunction with any dental tray (16) and gel method to accelerate the tooth whitening process. The SH primer of the invention is first applied to the facial surfaces of the teeth using an applicator such as a brush (14) or plastic strip (24) (FIGS. 1-5). After application of the SH primer (12), the patient places the dental tray (26) with peroxide tooth whitening gel over the dental arch. (See FIGS. 4, 5 and 5A) The primer (12) accelerates tooth whitening by disrupting the acquired pellicle and establishing a pH environment at facial surface and sub-surface of the teeth in which the primer of the invention was applied. After application of the dental tray (26 or 26a) with peroxide tooth whitening gel (22) to the dental arch, destabilization of the hydrogen peroxide gel ensues and hydroxyl and perhydroxyl free radicals are released as well as oxygen gas. When placed on the tooth surface, the primer acts to enhance and drive the reaction of peroxide tooth whitening gels to rapidly oxidize stains and accelerate the tooth whitening process. The SH primer begins to oxidize tooth stains and whiten teeth immediately after applying to the teeth.

All hypochlorite compositions (hypochlorite concentrations of between 0.1 and about 5.0 weight %) as described herein may further include a flavoring agent such as is described herein in U.S. Pat. No. 6,077,502 and in the described amounts of between about 0.001% and about 5% by weight. Other additives to the primer include but are not limited to sweetening agents, coolants, numbing agents, etc. as also described in U.S. Pat. No. 6,077,502.

The following examples are provided to be descriptive and illustrative only. They are not to be construed to be limiting in any manner.

As can be seen from the description herein, the present composition and method accelerates the tooth whitening process. The reduction in contact time with the tooth is reduced 10%, 20% or more compared to methods used to whiten teeth. Also acceptable levels of tooth whitening are achieved such that the frequency of tooth whitening events is concomitantly reduced by 10%, 20%, 30%, 50% or more. Tooth sensitivity and soft tissue irritation is reduced since exposure time to peroxide tooth whitening gels is shortened.

General

Unless otherwise specified the materials described herein are available from commercial sources in the U.S. and worldwide. The compositions and apparatus used for dental application are available from commercial dental supply companies.

It is understood by those skilled in this art that all components used and described herein are of dental, cosmetic, and/or pharmaceutical quality, purity and grade.

### EXAMPLE 1

### Primer and Prefilled Dental Tray

Another method of the tray-and-gel method uses a disposable dental tray pre-filled with peroxide tooth whitening gel. The pre-filled disposable dental tray can be purchased over-the-counter or dispensed to the patient by a dental professional. In a clinical study of 12 patients, the primer of the invention was used with a disposable dental tray pre-filled with 10% hydrogen peroxide gel. Each pre-filled disposable dental tray was individually packaged. In the professional dental setting, the SH primer was applied to the surfaces of the teeth after dental prophylaxis (teeth cleaning). Using the value oriented Vita shade guide, the shade of the maxillary right central incisor was recorded before the SH primer and pre-filled dental tray was applied to the facial surfaces of the teeth. Before placing the pre-filled dental tray with 10% hydrogen peroxide tooth whitening gel, a neutral pH fluoride solution was spayed into the dental tray. The pre-filled dental trays were placed over each dental arch and timed and recorded. After approximately 10 minutes, the trays were removed and the excess gel was rinsed from the teeth. Using the conventional valued oriented Vita shade guide, the tooth color of maxillary right central incisor (tooth number 8) was recorded.

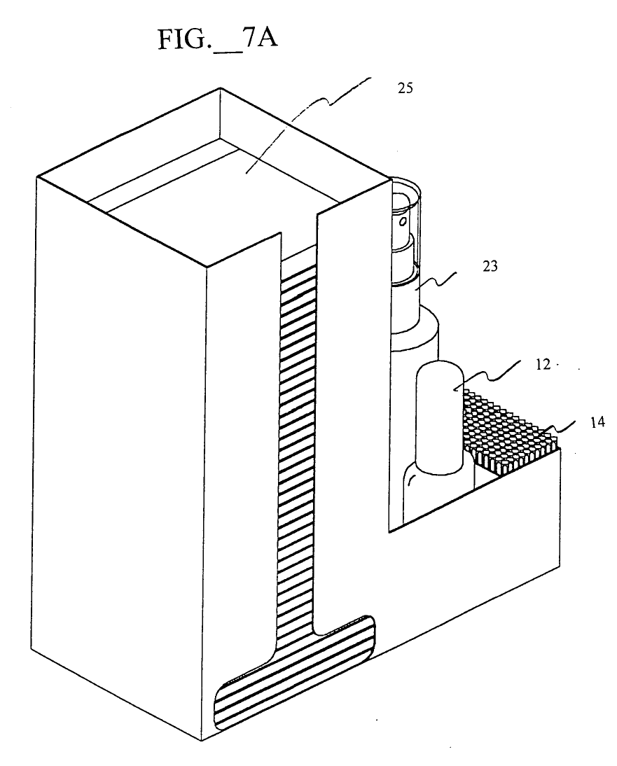

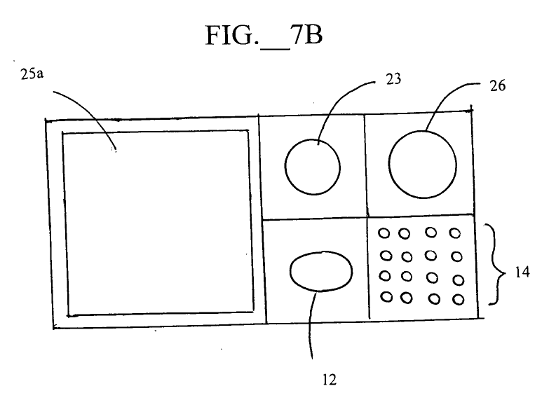

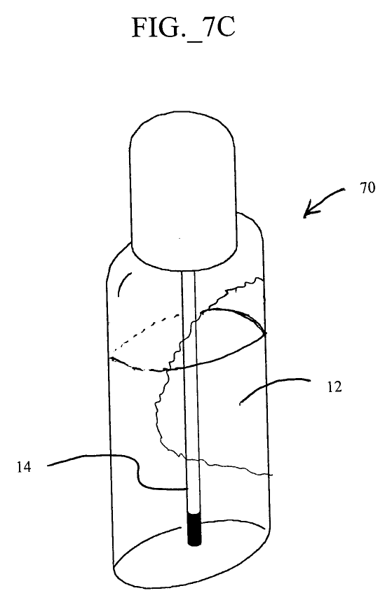

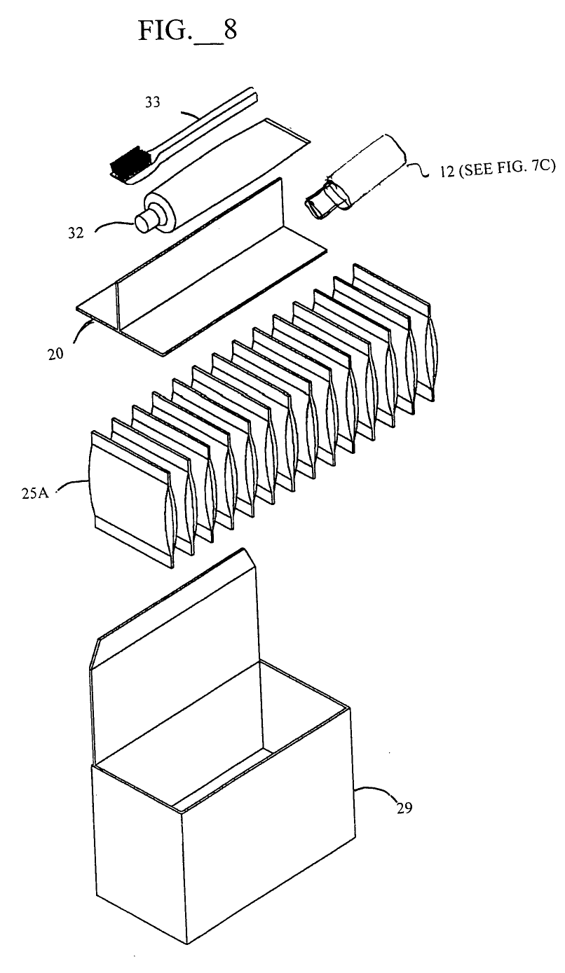

The teeth of 12 random patients, 7 females (58%) and 5 males (42%), were whitened using the SH primer of the invention in conjunction with a disposable tray-and-gel system using a neutral pH fluoride spray. Using the valued oriented Vita shade guide, the average shade of tooth number 8 was 8.9 (+/−4.4) before accelerated tooth whitening using the primer of the present invention. The average shade of tooth number 8 after tooth whitening using the primer of the present invention in combination with fluoride spray and the disposable pre-filled dental tray was 5.3 (+/−3.7). The average shade improvement of tooth number 8 was 3.7 (+/−3.4). The average treatment time in minutes was 11.2 (+/−3.6). Using primer of the present invention with the disposable dental tray pre-filled with 10% hydrogen peroxide, the average rate of whitening or shade improvement per minute is 0.58 (+/−0.46). Overall, the average percent shade improvement was found to be 43% (+/−37%). During the study, no sensitivity was reported during or after professional administration of the SH primer in conjunction with pre-filled dental trays with 10% hydrogen peroxide and neutral pH fluoride spray. (See FIG. 7.)

After application of the SH primer, the dental professional placed the dental pre-filled with 10% hydrogen peroxide over the dental arch for approximately 10 minutes. The primer accelerates tooth whitening by disrupting the acquired pellicle and establishing a pH environment at facial surface and sub-surface of the teeth in which the primer of the invention was applied. The primary staining zone of teeth is located in the acquired pellicle on the facial surface of the teeth and the facial enamel surface and facial enamel sub-surfaces of the teeth and into the facial surface of the dentin. After application of the dental tray pre-filled with peroxide tooth whitening gel to the dental arch, destabilization of the hydrogen peroxide gel ensues and hydroxyl and perhydroxyl free radicals are released as well as oxygen gas. When placed on the tooth surface, the primer acts to enhance and drive the reaction of peroxide tooth whitening gels to rapidly oxidize stains and accelerate the tooth whitening process. The SH primer begins to oxidize tooth stains and whiten teeth immediately after applying to the teeth.

At times the professional setting, it is difficult to diagnose the course of tooth stains and/or predict the treatment outcomes resulting from prescribed tooth whitening treatment regiments. Using the primer of the present invention with the disposable, pre-filled dental trays containing 10% hydrogen peroxide, it was discovered that patients could be categorized into response to the tooth whitening treatment using the primer. The results revealed that tooth whitening rates after professional administration of the primer and the pre-filled dental tray were observed to be slow, moderate, or quick. As such, patients are placed in the following categories according their response and overall shade improvement in about 10 minutes (Table 3):

TABLE 3
Categorizing patient tooth shade improvement after the accelerated tooth
whitening with the primer of the present invention used in combination
with a disposable dental tray pre-filled with 10% HP.

| | |
|---|---|
| Slow | Shade improvement ranging from 0 to 2 |
| | shades in about 10 minutes |
| Moderate | Shade improvement ranging from 3 to 5 |
| | shades in about 10 minutes |
| Quick | Shade improvement of 6 shades or greater |
| | in about 10 minutes |

Patient shade improved ranged from 0 to 10 shades. There were 5 (42%) patients that slowly (0 to 2 shade improvement) responded, 3 (25%) patients that moderately (3 to 5 shades) responded, and 4 (25%) patients that quickly (6 shades or greater) responded to tooth whitening using the primer to the present invention in combination with a disposable tray-and-gel method and fluoride spray.

Similarly, tooth whitening rates can also be grouped into the following categories according to percent shade improvement, which ranged from 0% to 100% (Table 4):

TABLE 4
Categorizing patient percent shade improvement after the accelerated tooth
whitening with the primer of the present invention used in combination
with a disposable dental tray pre-filled with 10% HP.

| | |
|---|---|
| Slow | Percent shade improvement ranging from 0% to 33% |
| Moderate | Percent shade improvement ranging from 34% to 66% |
| Quick | Percent shade improvement ranging from 67% to 100% |

According to percent shade improvement (Table 4), 5 (42%) patients responded slowly (0% to 33% shade improvement), 4 (33%) patients had a moderate (34% to 66% shade improvement) response, and 3 (25%) patients quickly (67% to 100% shade improvement) responded to tooth whitening using the primer to the present invention in combination with a disposable tray-and-gel method and fluoride spray.

Using the primer of the present invention coupled with disposable dental trays pre-filled with 10% hydrogen peroxide, dental professionals can triage patients undergoing tooth whitening. Dental professionals can prescribe professionally supervised tooth whitening treatments to patients that may provide the most effective results according to the patient's response to the primer of the present invention coupled with disposable pre-filled dental trays filled with 10% hydrogen peroxide. Dental professional generally have three professional options if patients elect to continue their professionally supervised tooth whitening being triaged with the primer of the present invention combined with the disposable dental tray pre-filled with 10% HP (Table 5):

TABLE 5
Professional tooth whitening treatment options for
patients after being triaged with the primer of the
present invention combined with the disposable
dental tray pre-filled with 10% HP with or without fluoride spray.

One hour chairside power bleaching
Custom fitted dental tray and whitening gel syringes or
Professionally supervised disposable at-home method, such as strips or
disposable tray-and-gel methods

From Tables 3 and 4, patient outcomes after being treated using with the primer of the present invention combined with the disposable dental tray pre-filled with 10% HP can be categorized as slow, moderate, and quick. Table 6 shows a flow chart for patients desiring to continue the tooth whitening process after outcomes using the primer of the present invention combined with the disposable dental tray pre-filled with 10% HP.

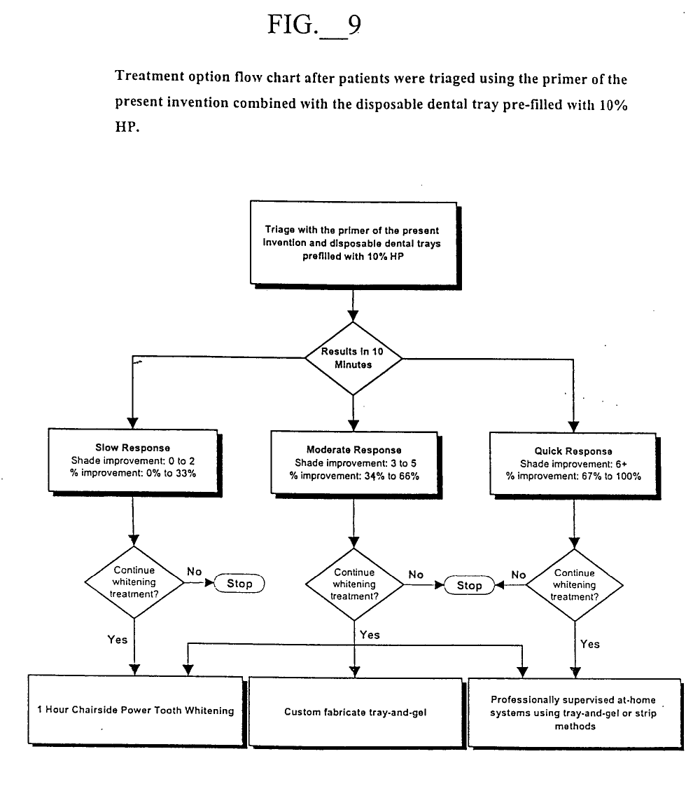

From Tables 3 and 4, approximately one-third of patients that were treated with the primer of the present invention combined with the disposable dental tray pre-filled with 10% HP had a slow response to tooth whitening, demonstrating a 0 to 2 shade improvement or 0 to 33% improvement in shade. For a number of contributing factors, these patients appear to have tooth staining which responds slowly to peroxide based tooth whitening. If the patient desires to continue the whitening process, it is recommended that they undergo in-office chair side power bleaching using 20% to 35% hydrogen peroxide gel. FIG. 9 shows the diagnosis and the options available to the patient.

From Tables 3 and 4, approximately one-third of patients that were treated using the primer of the present invention combined with the disposable dental tray pre-filled with 10% HP had a moderate response to tooth whitening, demonstrating a 3 to 5 shade improvement, or 34% to 66% improvement in shade. Clinically, moderately responding patients have three alternatives to continue their whitening process, namely one hour chair side power bleaching, custom tray-and-gel tooth whitening, or professionally supervised at-home system using disposable tray-and-gel or strip methods.

From Tables 3 and 4, approximately one-third of patients that were treated using the primer of the present invention combined with the disposable dental tray pre-filled with 10% HP had a quick response to tooth whitening, demonstrating at least a 6 shade or more improvement, or 67% to 100% improvement in shade. It is recommended that quickly responding patients are dispensed professionally supervised at-home systems using disposable tray-and-gel or strip methods.

### EXAMPLE 2

### Primer and Peroxide

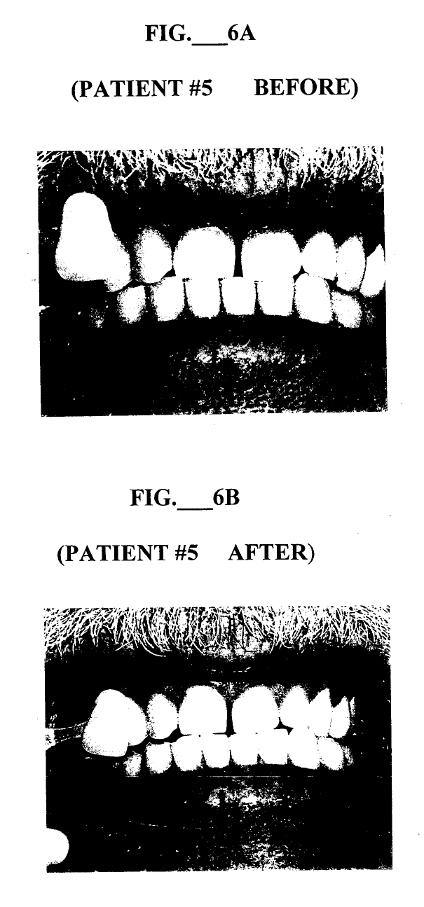

One patient was treated with primer (SH) 3.2% composition for less than 1 minute followed by peroxide treatment 10% HP gel for 10 minutes. After treatment and rinsing the primer and peroxide, the teeth and the spaces between the teeth were visibly whiter. FIG. 6A is the before whitening photo (taken just prior to the procedure) and is compared with FIG. 6B as the photo taken after of completion of whitening. This patient has a two (2) shade improvement on the surface of the teeth. Although the patient had a slow response, there was significant shade improvement in the interproximal area of the anterior teeth.

### EXAMPLE 3

### In Office Kit

FIG. 7A is a schematic representation of a whitening kit to be used by a professional. Included in the dental kit is primer (12), primer application brushes (14), disposable cups (not shown), optional fluoride solution (23) and intrinsically packaged disposable dental trays pre-filled with HP gel (25). The instructions are not shown. FIG. 7B is a top plan view of the kit of FIG. 7A showing these described articles and the disposable cups (27), primer (12), brushes (14), disposable dental tray and gel (25a), optional fluoride (23), etc. FIG. 7C is a cutaway schematic representation of the individual container (70) of the primer.

### EXAMPLE 4

### Consumer Kit

FIG. 8 is a schematic representation of this whitening kit. It is to be used by the patient or consumer. Included is a whitening kit (e.g. any OTC whitener), primer (12), with application brushes (14) (if optional) (see FIG. 7C), individually packaged disposable dental trays (25A) pre-filled with HP gel, separator (20) is a package separator, desensitizing tooth paste (with or without fluoride) (32), tooth brush (33) and the outside container (29). The instructions are not shown and the fluoride solution is optional.

### EXAMPLE 5

### Primer/Whitener Kits

(a) In FIG. 7C, the primer (12) above comprising hypochlorite in some situations is sold as a stand-alone kit with an applicator (e.g. a brush) (14) and instructions. This primer has a dual function as the primer to break up the pellicle and to simultaneously and independently cause whitening of the tooth at the same time. In some instances the customer will pre-treat the tooth with a dental fluoride solution or dentifrice (prior to the primer) or treat the tooth with a dental fluoride solution or dentifrice after application of the primer. These treatment components are then removed after whitening is completed.

(b) The kit of Example 5(a) further includes a dental fluoride treatment composition and an applicator.

(c) The kit of Example 5(a) or 5(b) further includes a tooth whitening composition comprising peroxide with or without a method of applying peroxide tooth whitening composition to the dental arch.

(d) The kit of Example 5(a), 5(b) or 5(c) further includes a desensitizing toothpaste and optionally a toothbrush.

### EXAMPLE 6

### Primer/Flavoring Composition

(a) The primer as a stand-alone 2 wt % solution of a aqueous sodium hypochloride also contains 1% of a commercially available oil of peppermint flavor. This composition is stable in storage for at least 90 days and longer and is more easily tolerated by the patient for treatment of 15-30 minutes. The tooth whitening improvement observed is 2 or more shades as is described herein. (See FIG. 7C.)

(b) Example 6(a) is repeated except that the oil of peppermint is replaced by a stoichcometrically equivalent amount of oil of spearmint. The experimental results and tooth whitening are similar.

While only a few embodiments of the invention have been shown and described herein, it will be apparent to those skilled in the art that various modifications and changes can be made in the tooth whitening method, compositions, diagnosis method, various kits and the like without departing from the spirit and scope of the present invention. All such modifications and changes coming with the scope of the appended claims are intended to be carried out thereby.

1. An improved composition for whitening a tooth surface, which composition comprises:
(a) a stable high pH primer solution itself comprising hypochlorite having a pH greater than about 8.5 to about 13; and
(b) a tooth whitening composition itself comprising peroxide;
wherein component (a) and component (b) are contacted with each other on and comprise an active composition on said surface of a tooth.

2. The improved composition of claim 1 wherein in step (a) the primer has a pH of between about 8.5 and 12.

3. The improved composition of claim 1 wherein the hypochlorite is selected from the group consisting of sodium hypochlorite, potassium hypochlorite, calcium hypochlorite, barium hypochlorite and combinations thereof.

4. The improved composition of claim 1 wherein the peroxide is selected from the group consisting of hydrogen peroxide, sodium peroxide, potassium peroxide, carbamide peroxide, sodium perborate, sodium percarbonate and combinations thereof.

5. The improved composition of claim 1 wherein the peroxide is hydrogen peroxide.

6. The improved composition of claim 1 wherein the hypochlorite is sodium hypochlorite.

7. An improved method for whitening the surface of a tooth, which method comprises:
(a) contacting the surface of a tooth with a stable high pH primer solution itself comprising hypochlorite having a pH greater than about 8.5 to about 13;
(b) contacting the pH primer solution on the surface of the tooth with a tooth whitening composition itself comprising peroxide; and
(c) removing after an appropriate contact and reaction time, said pH primer solution and said tooth whitening composition.

8. The improved method of claim 7 wherein:
in step (a) the hypochlorite is selected from the group consisting of sodium hypochlorite, potassium hypochlorite, calcium hypochlorite, barium hypochlorite and combinations thereof.

9. The improved method of claim 7 wherein:
in step (b) the peroxide is selected from hydrogen peroxide, sodium peroxide, potassium peroxide, carbamide peroxide, sodium perborate, sodium percarbonate and combinations thereof.

10. The improved method of claim 7 wherein the peroxide comprises hydrogen peroxide.

11. The improved method of claim 7 wherein the hypochlorite comprises sodium hypochlorite.

12. The improved method of claim 7 wherein the method further includes:
(a1) contacting,
(i) before step (a) or
(ii) between step (a) and step (b),
the tooth surface and the applied pH primer solution with an aqueous, foam or gel fluoride dental treatment composition, and
(b1) after step (b), removing said fluoride dental treatment composition separately or concurrently in step (c).

13. The improved method of claim 12 wherein:
in step (a) the hypochlorite is selected from the group consisting of sodium hypochlorite, potassium hypochlorite, calcium hypochlorite, barium hypochlorite and combinations thereof;
in step (a1) the fluoride composition is applied between step (a) and (b); and
in step (b) the peroxide is selected from the group consisting of hydrogen peroxide, sodium peroxide, potassium peroxide, carbamide peroxide and combinations thereof.

14. A kit for the improved whitening of tooth, which kit comprises:
(a) a composition of a stable high pH primer-whitener solution itself comprising hypochlorite having a pH of greater than about 8.5 up to about 13,
(b) means for applying said primer-whitener solution to the surface of said tooth; and
(c) instructions describing said improved tooth whitening process.

15. The kit of claim 14 which further includes:
(a) a second tooth whitening composition itself comprising a peroxide, and
(b) means for applying said second tooth whitening composition to said tooth.

16. A kit for improved whitening of a tooth, which kit comprises:
(a) a composition of a stable high pH primer solution itself comprising hypochlorite having a pH of greater than about 8.5 up to about 13;
(b) means for applying said pH primer solution to the surface of said tooth;
(c) a tooth whitening composition itself comprising a peroxide;
(d) means for applying said tooth whitening composition to said tooth;
(e) optionally a dental fluoride treatment composition;
(f) optionally means for applying said optional dental fluoride treatment composition to the surface of said tooth; and
(g) instructions describing said improved tooth whitening process.

17. The kit of claim 16 wherein:
components (e) and (f) are not present.

18. The kit of claim 16 wherein components (e) and (f) are present.

19. An improved method for diagnosis and determination of tooth whitening options, which method comprises
(a) performing the tooth whitening of claim 67;
(b) evaluating the results of step (a); and
(c) providing specific tooth whitening treatment options to the patient.

20. The improved method of claim 19 wherein the step (c) the treatment options include stopping tooth whitening treatments, chair side power tooth whitening, custom fabricate a tray-and-gel, tray and gel strip, supervised at-home methods and over-the-counter kits.

21. An improved composition for whitening a tooth surface, which composition comprises:
(a) a stable high pH primer solution itself comprising hypochlorite having a pH greater than about 8.5 to about 13 which is present in a concentration of between about 0.1 and 5 weight % of the total composition; and
(b) a flavoring agent in the amount of between about 0.001 and 5% by weight of the total composition; and are mixed with each other and comprise an active composition on said surface of a tooth.

22. The improved composition of claim 21 wherein the flavoring agent is selected from the group consisting of, but is not limited to mint, oil of wintergreen, oil of peppermint, oil of spearmint, clove bund oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal, and mixtures thereof.
